(19) United States
(12) Patent Application Publication
(10) Pub. No.: US 2019/0046801 A1
(43) Pub. Date: Feb. 14, 2019

(54) Tremor Control Mouth Piece

(71) Applicant: Kimberly K. Zamorano, Oklahoma City, OK (US)

(72) Inventor: Kimberly K. Zamorano, Oklahoma City, OK (US)

(21) Appl. No.: 15/674,997

(22) Filed: Aug. 11, 2017

(51) Int. Cl. A61N 1/36; A61C 7/08

(52) U.S. Cl. CPC A61N 1/36067; A61H 2201/5071; A61N 1/36125; A61C 7/08

(57) ABSTRACT

A tremor control mouth piece comprising at least one pressure applicator contacting and applying pressure to at least one appropriate pressure point on the lingual aspect of a patient's maxilla, a structure fitting inside and engaging the lingual aspect of the patients maxilla and retaining the pressure applicator; and a connector for removably affixing the mouth piece to at least one engaging surface inside the mouth, whereby a structure is placed and secured inside the mouth and the appropriate pressure is applied to a pre-determined pressure point thereby reducing body tremors.

### CROSS-REFERENCE TO RELATED APPLICATIONS

None.

### FIELD OF THE INVENTION

The invention generally relates to an apparatus placed and secured in the mouth to assist with controlling and reducing the signs of neurogenic tremors and neurological spastic hemiparesis. In particular, the invention relates to an apparatus for controlling and reducing tremors by placing pressure on a pre-determined pressure point in the lingual aspect of the maxilla, where the removable apparatus is positioned and secured inside the mouth of a person with tremors and a pad engages the pre-determined pressure points to provide the minimal amount of pressure required to facilitate while decreasing the severity and frequency of tremors.

### BACKGROUND

The mental, social and physical stigma of people with self-uncontrollable Autonomic Nervous System Imbalances of autonomic flexibility in the Autonomic Dysreflexia or Autonomic Hyperreflexia systems manifesting as trembling and quivering, either internally or externally, is at times insurmountable. Diagnosis and current treatments which relate to neurological disorders without the use of medications or surgery, which could manifest as unwarranted side effects, compounds the stress of living with the imbalance even further. Addressing the disruption of daily living skills such as: eating, drinking, dressing, grooming, and public appearances is critical to a better quality of life. Giving a neurologically impaired person the power to maintain emotional, mental, physical health and increasing the quality of human life, without use of chemicals and their unwarranted side effect, risks of a surgical procedures and possible complications, and other solutions that have their own associated problems is highly desirable.

Physicians have attempted to find relief for persons afflicted with tremors with medications, hypnosis or shock therapy, and these solutions work to varying degrees. Medications may present undesirable side effects that make the solution worse than the tremor itself. Through hypnosis, most psychiatrists focus on the trance state brought on by intentional relaxation and focusing exercises. Intentional relaxation may be unobtainable for Restless Leg Syndrome patients as many tremble while resting or asleep. Some people have difficulty focusing on a task at hand and retaining a focused relaxation exercises using the same muscle group at the same time. Electroshock therapy (ECT) or “convulsive therapy” has been used since 1938 to treat certain forms of mental illness by causing convulsive seizures within the brain. Administered four to twelve times in a hospital setting, partial memory loss sometimes occurs with treatment, and outcome is not certain until recovery. Frequently facilities and insurance companies have deemed ECT as a barbaric form of treatment and refuse to engage in this form of therapy. Sweden frequently treats Parkinson's disease with a surgical procedure in which the patient's brain is re-sectioned into distinct hemispheres; again, outcome is not certain until recovery. Deep Brain Stimulation (DBS) is a surgical procedure which attaches a stimulator deep in the mid-brain. The high risk, invasive surgery insures lengthy rehabilitation without a guarantee of the successful resolution of the neurological impairments or the patient's ability to socially re-integrate. DBS is not genuinely used for patients affected by relatively mild tremors. A Vegus Nerve Stimulator (VNS) device implanted subdural left subclavicular, sends a burst shock, on a timed interval, which is received by the brain to reduce or stop the tremor. However, after two years, the solution has found to relieve only 49% of the patients, and some don't respond to VNS therapy at all. Each of the aforementioned solutions has their own possible side effects, complications, or unforeseen outcomes.

### SUMMARY OF THE INVENTION

The present invention overcomes the shortcomings by providing a tremor control mouth piece when inserted into a patient's mouth, when they choose, during waking hours, which applies pressure to a pre-determined pressure point to reduce the neurogenic tremors. The tremor control mouth piece allows a patient that is a victim of neurogenic tremors, the ability to control their limbs without medications, diet restrictions, orthopedic braces, or social embarrassment. Assisting the patient's ease of comfort for daily living skills such as: eating, personal hygiene, social interaction and recreation activities is obtainable. The apparatus may consist of a mouth piece having a structure to engage the upper part of the mouth, a pressure applicator and connector to removeably affix the mouth piece to the upper part of the mouth.

There have thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out several purposes of the present invention. Additional benefits and advantages of the present invention will become apparent to those skilled in the art to which the present invention relates from the subsequent description of the preferred embodiment and the appended claims, taken in conjunction with the accompanying drawings. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### DESCRIPTION OF THE DRAWINGS

FIG. 1 is a perspective view of the upper portion of the mouth and a retainer according to the present invention to engage the upper portion.

FIG. 2 is an underside view of the upper portion of the mouth and retainer according to the present invention to engage the upper portion.

FIG. 3 is a perspective view of a denture with a pad.

FIG. 4 is a view of the upper portion of the mouth with tremor control mouth piece engaged.

FIG. 5 is an illustration of synaptic process.

### DETAILED DESCRIPTION OF THE INVENTION

The CranioSacral system consists of the membranes and bones which surround and protect the brain and spinal cord which encourages and facilitates circulation of spinal fluid, from the jaw and cranial vault or head down to the sacrum and coccyx or tailbone. CranioSacral Therapy (CST) is used to detect and correct imbalances in the cranioscral system's rhythm, which may be associated with sensory, motor and neurological dysfunction. A CST practitioner uses a gentle, alternative, light touch manual therapy, approach that is based on the controversial theory of detecting and correcting imbalances in the CranioSacral system by encouraging the release of tension within the Dura and Pia mater of the spinal column so every other system in the body can relax, self-correct, and free itself of pain and discomfort. Current understanding is certain physical problems may be caused by disturbances in the movement or lack of movement in the cranial structures and normal rhythmic flow of the craniosacral system. A ten-step protocol was developed by Physicians for CST Practioner's use in treating the cranioSacral system for clients worldwide. Additional steps consisting of oral light pressure movements, added by the inventor to the ten-step protocol are used to identify the specific trigger points for the patient. Those additional steps to the CST to identify the trigger point are protected as trade secrets. Once the trigger point is identified a tremor control device may be crafted for the patient. Once the tremor control device is received, a final fitting is performed and the pressure applicator 120 is adjusted to provide the desired pressure to the trigger point. Currently, the present invention is designed to address tremors, however, the present invention may be able to affect other medical conditions in the same way by applying pressure to other trigger points associated with particular medical conditions.

Observations have shown, a small amount of pressure (1-10 gf/cm²) applied by the raised area on the lingual aspect of the maxilla near the central incisor, on upper gingiva will occlude the CA(++) channel between the synaptic cleft dendrite and axon terminal causing the neurotransmitter—receptor an invalid connection transfer energy space near the maxillary division of the Trigeminal Nerve (V). Thus, an interception of spastic muscle signals to the Pons in the brain stem for the autonomic nervous system to react to manifesting noticeably fewer or diminished limb tremors. Several of the observations are discussed below.

Nov. 2, 2015 a elderly patient had external spastic tremors related to a diagnosis of Parkinson's disease. The patient received CST and also received the additional oral manipulations to identify the trigger point for this particular patient. After locating the trigger point the therapist asked the patient to place their tongue on the trigger point and apply pressure. After applying pressure, the external spastic tremors related to Parkinson's disease visibly stopped or diminished as a result of the maintaining constant pressure on that point.

Mar. 7, 2015 an elderly patient having familial bilateral upper extremity neurogenic tremors had displayed tremors for over 30 years. The tremors were so great the patient could not get a spoonful of peas to their mouth. The patient received CST and the additional oral manipulations to identify the trigger point for this particular patient. The therapist applied pressure digitally to the trigger point and the tremors were diminished to a level that was acceptable to the patient.

March 2016, a young patient with extremity neurogenic tremors that had been caused by fever of unknown origin when the patient was living abroad. The patient received CST and the additional oral manipulations to identify the trigger point for this particular patient. After locating the trigger point the therapist asked the patient to place their tongue on the trigger point and apply pressure. After applying pressure, the external spastic hemiparesis tremors were no longer present when the pressure remained. For the first time in their life the patient was able to extend their right arm to retrieve a full bottle of water without tremors shaking and spilling the water on their self or the floor.

June 2016, a patient with excruciatingly painful bilateral foot neuropathy and restless leg syndrome, tried . . . pharmaceuticals, acupuncture, physical therapy and has been seen at the Mayo Clinic but nothing seemed to help. The patient received CST and the additional oral manipulations to identify the trigger point for this particular patient. The patient could press their tongue lightly against the identified pressure point and diminish the pain in their feet, and the legs were noticeably able to relax, without spastic movement.

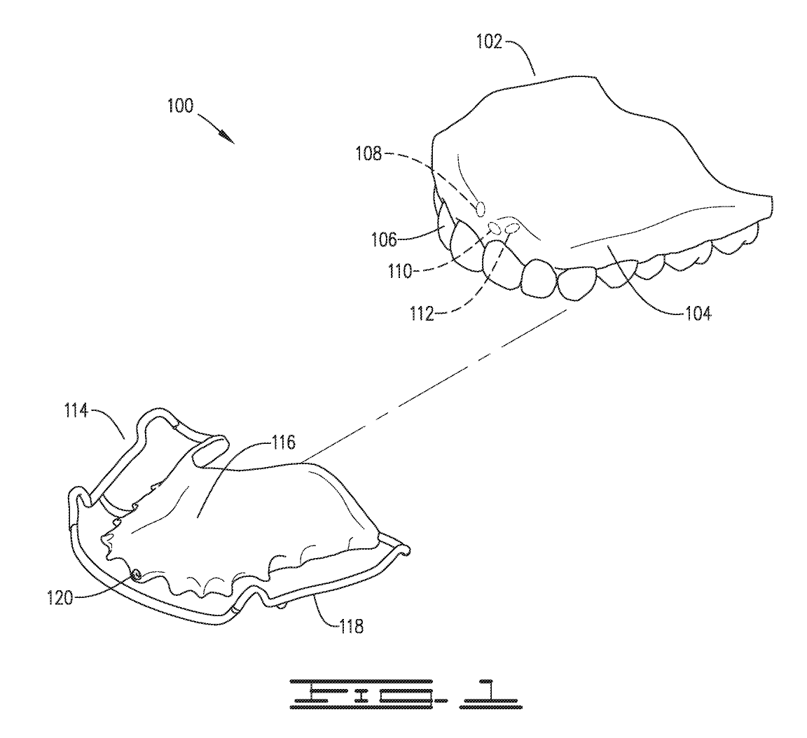

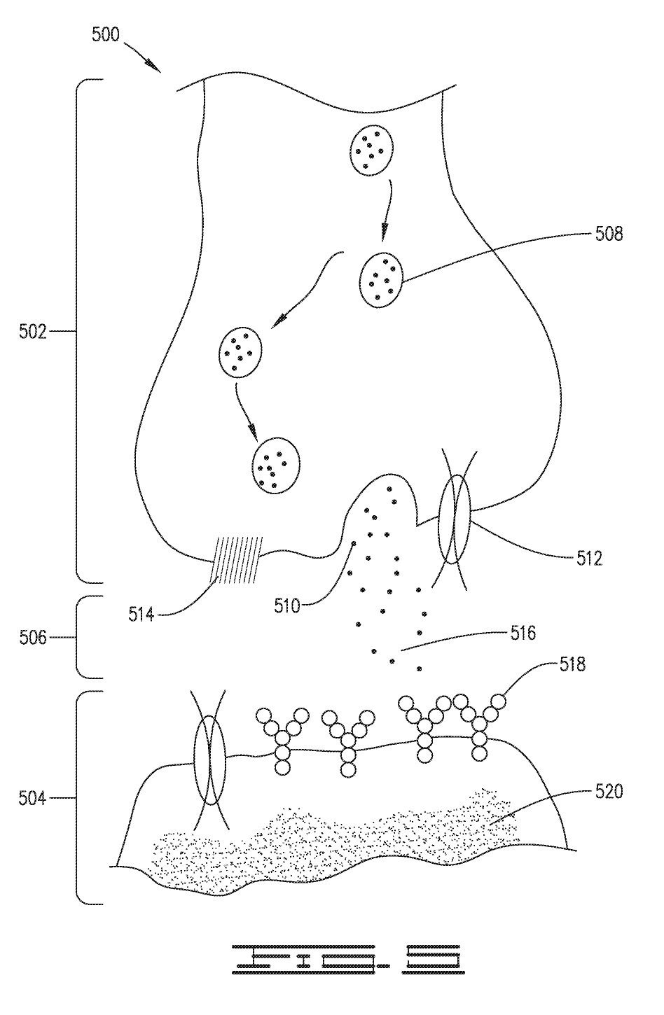

FIG. 1 is a exploded view of the upper mouth from the top with a retainer detached 100. This view of the upper mouth 102 shows the gingivae 104; teeth, specifically the incisors teeth 106 with a left trigger point 108, a middle trigger 110 and a right trigger point 112. Hereinafter, the left trigger point 108, the middle trigger 110 and the right trigger point 112 will each, for simplification purposes, be referred to as a “trigger point” for the reasons discussed below. Typically, one of the trigger points 108, 110, 112 may be engaged to affect the neurogenic tremors in a specific patient. Current anecdotal evidence does not suggest any added benefit by engaging multiple trigger points 108, 110, 112 simultaneously. Every patient is different and the trigger points 108, 110, 112 and pressure required to induce a therapeutic effect for an individual patient may be determined through a modified CranioSacral treatment protocol. Once this protocol has been completed, the specific trigger point 108, 110, 112 that will reduce, minimize, or possibly eliminate the neurogenic tremors may have been identified along with the appropriate pressure needed to engage the trigger point 108, 110, 112. With the appropriate trigger point 108, 110, 112 and pressure identified for a patient, one skilled in the art may create a mouth piece for use inside the mouth for the specific purpose of tremor control (see FIG. 4) or may modify other dental devices including, but not limited to, dentures, mouth guard, retainers, implants, splint, night guard, braces, and bridges previously fitted for by the patient to apply pressure the appropriate trigger point 108, 110, 112. The creation of the mouth piece may use the common methods used to create the aforementioned dental devices such as molding but may also include, but is not limited to, the use of 3-dimensional printing to create a new device or modify an existing device. Additionally, the mouth piece may be man-made or computer constructed from materials including but not limited to resin, ceramic, metal, poly (vinyl acetate-ethylene) copolymer thermoplastic; polyurethane; laminated thermoplastic; thermo-polymer, acrylic, polypropylene or polyvinylchloride material, co-polymer, and polymer.

An embodiment of the invention is a retainer 114 that may comprise a base plate 116, a metal wire 118, and a pressure applicator 120. One skilled in the art would be aware that the base plates for these types of retainers may be acrylic or created using vacuum formed polypropylene or polyvinylchloride materials. The pressure applicator 120 may be designed to apply the appropriate pressure to the trigger points 108, 110, 112 determined to have a desired effect on the neurogenic tremors for that patient. For illustration purposes, the right trigger point 112 will be used but the design and application would be the same for the left trigger point 108 and the middle trigger point 110. This pressure applicator 120 may be designed to apply 1 to 15 gf/cm² of pressure to the appropriate trigger point 112 identified during the earlier multistep craniosacral protocol. A retainer 114 may preferably be designed with the pressure applicator 120 in a corresponding position to the determined trigger point 112 to apply an appropriate amount of pressure to the trigger point 112 thereby reducing, minimizing, or possibly eliminating the neurogenic tremors. The retainer 114 may be adjusted to apply this desired pressure by adding different thicknesses of the pressure applicator 120 to achieve the desired pressure. The pressure applicator 120 material may include, but is not limited to polymers, copolymers, metal, rubber, adhesives, and cloth. The pressure applicator 120 material may be layered or varied in thickness to change the level of pressure applied to the trigger point 112. By varying the thickness and providing the ability to layer, one skilled in the art can fine tune the patients pressure to the minimal required to have a therapeutic effect. Any more pressure than is required to have the therapeutic effect, may desensitize the trigger point 112.

Typically, this retainer 114 may be removable where a patient may wear the retainer 114 to provide the effect of reducing, minimizing, or eliminating the neurogenic tremors as they are out in public and as to minimize their embarrassment. The removable retainers 114 are the preferred embodiment because a fixed retainer applying a consistent pressure to the determined pressure point may over time likely desensitize that trigger point 108, 110, 112 and allow the neurogenic tremors to increase and or return to the patient.

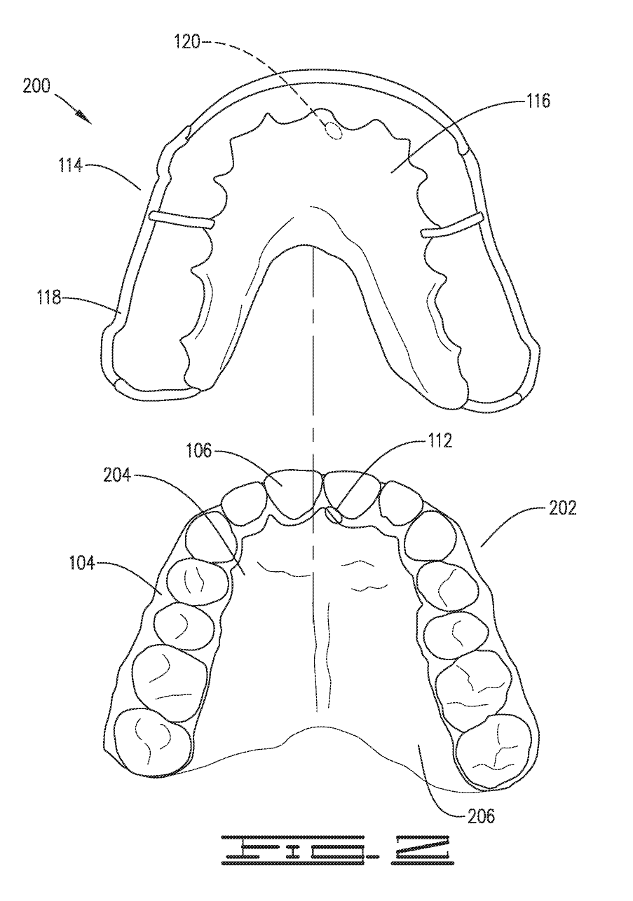

FIG. 2 is an underside view of the upper mouth with a retainer detached 200. This view shows the trigger point, 112, next to the incisor, 106. The other trigger points, as described earlier, the left trigger point, 108, the middle trigger point, 110, are also located near the central incisors, 106. Additionally, this view illustrates the relational location to the hard palate, 204 and the soft palate, 206, which provides a reference to the trigger points 108, 110, 112, that is, in this illustration, medial to the right central incisor, 106. When the retainer, 114, is placed inside the mouth and over the upper teeth the pressure applicator 120, applies direct pressure to the example trigger point, 112, to provide the pressure determined earlier with the craniosacral protocol.

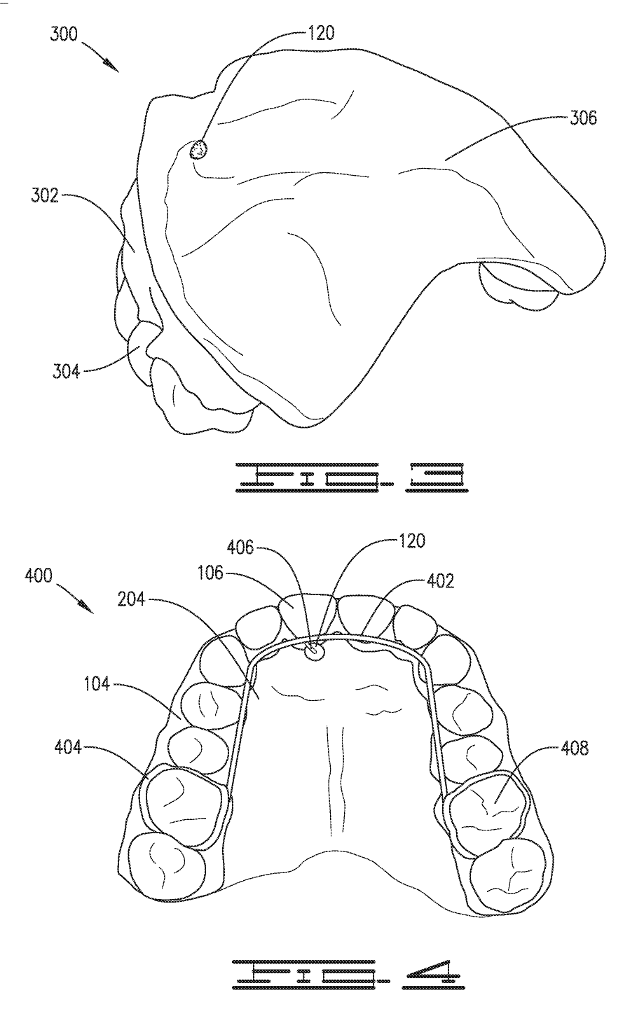

FIG. 3 illustrates a perspective view of another embodiment of the invention using a complete upper denture 300 with the pressure applicator 120. In this embodiment, the complete upper denture 300 comprises the simulated gingivae 302 and simulated teeth 304 and has a denture upper surface 306. One skilled in the art in creating an upper denture 300, a denturist, may form the upper denture 300 with the pressure applicator 120 as part of the upper denture 300 itself. Additionally, the upper denture 300 may be created with thicker pressure applicator 120 that would allow it to be adjusted to acquire the desired pressure for the patient once the dentures are received. The excess material may be removed by one skilled in the art to create the desired amount of pressure to provide the therapeutic properties to reduce, minimize or eliminate the neurogenic tremors. Additionally, a recess in pressure applicator 120 area or the patient's normal contour area will allow one skilled in the art to apply material to the denture 300 to build up the pressure applicator 120 to achieve the desired therapeutic results. The recess may provide for better adhesion of the pressure applicator 120.

FIG. 4 is a view of the upper portion of the mouth with a tremor control device 400 affixed to the upper teeth. The tremor control device 400 may comprise an upper fitted structure 402, a tooth anchor 404, and a pad connector 406 and a pressure applicator 120. The upper fitted structure 402 fits securely inside the mouth and is anchored to molars 408 on opposing sides of the mouth to minimize or prevent movement of the pressure applicator 120. The tremor control device 400 is positioned such that the pad connector 406 and the pressure applicator 120 apply pressure to the trigger point 108, 110, 112. The pad connector 406 may be mechanically adjusted to adjust for changes in the trigger point 108, 110, 112 and may be adjusted to also increase or decrease the pressure of the trigger point 108, 110, 112. As described above, the pressure applicator 120 may also be adjusted by adding or removing layers of material to the pressure applicator 120. For instance, the pad connector 406 may be adjusted to apply less pressure than is required to provide therapeutic effect but then in combination, additional material may then be added to the pressure applicator to achieve the desired pressure and therapeutic effect. This combination may reduce the overall cost by reducing the amount of material need for the tremor control device 400.

Another embodiment of the tremor control device 400 uses a thermoplastic mouth guard wherein the thermoplastic is heated per the instructions, placed over the teeth and then fitted to such teeth. This type of mouth guard is commonly used in sports to protect athletes particularly in contact sports. Once the fitting of the mouth guard has occurred and it is secure then a pressure applicator 120 with the correct layering to provide the appropriate pressure to the trigger point 108, 110, 112 may be added to the thermoplastic mouth guard. This allows for a cheaper alternative as compared to modified dental device. For aesthetic purposes, the mouth guard may be trimmed such that it is invisible from the outside while keeping the pressure applicator 120 on the trigger point 108, 110, 112.

FIG. 5 shows an illustration of the chemical synaptic process 500. The trigger point 108, 110, 112 may be one of the synapses and the modified CranioSacral treatment may identify the trigger points 108, 110, 112 for some neurogenic tremors. The axon terminal 502 of the neuron is separated from the synaptic cleft dendrite 504 of another cell by the synaptic cleft 506. The synaptic vesicles 508 containing the neurotransmitters 510 flows down through the axon terminal 502 where the synaptic vesicle 508 attaches to the presynaptic membrane. The voltage gated calcium CA(++) channel 514 triggers the release of the neurotransmitters 510 from the synaptic vesicles 508 into the synaptic cleft 506 where they are transported through the membrane via neurotransmitter transporter 512. Once attached the neurotransmitters 510 are released, they flow through the synaptic cleft 506 through the voltage (−) channel 516 to the receptors 518 and postsynaptic density 520 in the synaptic cleft dendrite 504 thus transporting the electrical information in the axon terminal 502 to the synaptic cleft dendrite 504 which is repeated to transfer signals to and from the brain and to and from other parts of the body. The pressure applicator acts to compress the synaptic cleft dendrite 504 and the axon terminal 502. Observations have shown, external tremors diminish or stop as long as 3-5 gf/cm² of pressure is applied to the upper mouth 102 near the alar cartilages to occlude or obstruct the channel 506. The pressure applied could range from 1-10 gf/cm² This occlusion of the channel 506 between the synaptic cleft dendrite 504 and axon terminal 502 near the distal edge of the medial head of the trigeminal nerve interrupts the electro-chemical synaptic process 500 nerve impulse/response loop received by the brain stem thereby reducing, minimizing, or eliminating the neurogenic tremors.

Having thus described the invention, I claim:

1. A tremor control mouth piece comprising:
a. at least one pressure applicator contacting and applying pressure to at least one appropriate pressure point on a lingual aspect of a patient's maxilla, where the points selected are found to reduce the patient's tremors;
b. a structure fitting inside and engaging the lingual aspect of the patient's maxilla and retaining the pressure applicator; and
c. a connector for removably affixing the mouth piece to at least one engaging surface inside the mouth,
whereby a structure is placed and secured inside the mouth and appropriate pressure is applied to a pre-determined pressure point thereby reducing body tremors.

2. The mouth piece of claim 1, where the structure is selected from dentures, mouth guard, retainers, implants, splint, night guard, braces, and bridges.

3. The mouth piece of claim 1, where the mouth piece is comprised of materials selected from resin, ceramic, metal, polyvinyl acetate-ethylene copolymer thermoplastic, polyurethane, seaweed, laminated thermoplastic, thermo-polymer, acrylic, polypropylene or polyvinylchloride material, co-polymer, and polymer.

4. The mouth piece of claim 1, where the connector is selected from pressure, friction, mechanical, and adhesive.

5. An mouth piece for controlling tremors for a patient, the apparatus comprising:
a. an upper fitted structure;
b. a low pressure area for removably engaging an interior upper surface of the mouth;
c. at least one pad to apply the desired pressure to at least one desired point on lingual aspect of the maxilla; and
d. a pad connector;
whereby the mouth piece is placed and secured inside the mouth and the pad applies the desired pressure to a pre-determined pressure point thereby reducing body tremors.

6. The mouth piece of claim 5, where the pad is selected from polymers, metal, rubber, adhesives, and cloth.

7. The mouth piece of claim 5, where the pads may be varied in thickness to adjust the amount of pressure applied.

8. The mouth piece of claim 5, where the pad may be layered to achieve the appropriate amount of pressure.

9. The mouth piece of claim 5, where the pad connector is selected from mechanical, and adhesive connection.

10. The mouth piece of claim 5, where the pad connector applies in a range of 1 to 10 grams/cm² of pressure.
